(19) United States
(12) Patent Application Publication
(10) Pub. No.: US 2024/0350214 A1
(43) Pub. Date: Oct. 24, 2024

(54) MASTER CONTROLLER DEVICE FOR MEDICAL OR SURGICAL TELEOPERATION

(71) Applicant: MEDICAL MICROINSTRUMENTS, INC., Wilmington, DE (US)

(72) Inventors: Federico MONTAGNANI, Pisa (IT); Nicola PINESCHI, Pisa (IT); Massimiliano SIMI, Pisa (IT); Alexander Richard JOSEPH, Cambridge (GB); Andrew Duncan PIDGEON, Cambridge (GB); Samuel John DOYLE, Cambridge (GB); Benjamin David HOPKINS, Cambridge (GB); Niven CURTIS-WOODCOCK, Cambridge (GB); Christopher Edward HARM, Cambridge (GB); Alexander John MARET, Cambridge (GB)

(21) Appl. No.: 18/639,681

(22) Filed: Apr. 18, 2024

(30) Foreign Application Priority Data
Apr. 21, 2023 (IT) 102023000007863

(51) Int. Cl. A61B 34/37; A61B 34/00; A61B 34/20; A61B 34/30; A61B 46/10

(52) U.S. Cl. CPC A61B 34/37; A61B 34/20; A61B 34/74; A61B 46/10; A61B 2034/2046; A61B 2034/305

(57) ABSTRACT

A master controller device includes a control gripper to control at least the open/close degree of freedom of a slave surgical instrument. The control gripper has a first interface portion and a second interface portion for the fingers of a user, wherein the first interface portion is opposite to the second interface portion. Each interface portion includes a cavity thereof formed by a respective conically-developing surface. The controller device can further include a handle which forms the handle of the master controller device, wherein the handle has a body which forms at least one seat with a surface thereof to rest on the back of a user's hand.

### CROSS-REFERENCE TO RELATED APPLICATIONS

The present application claims priority to Italian Patent Application No. 102023000007863 filed on Apr. 21, 2023, which application is incorporated herein by reference in its entirety.

### FIELD OF THE INVENTION

The present invention relates to a master controller device.

The master controller device according to the invention is designed for a robotic surgical teleoperation system, as well as for a surgical teleoperation simulator.

The master controller device according to the invention is particularly suitable, although not uniquely intended, for use as a controller device of the type not mechanically constrained to the operative console.

### BACKGROUND ART

Robotic surgery apparatuses are generally known in the art and typically comprise a central robotic tower or a cart and one or more robotic arms extending from the tower/cart. Each arm comprises a motorized positioning and orientation system (usually referred to as a manipulator) for moving a surgical instrument distally attachable thereto, in order to perform surgical procedures on a patient.

Typically, the “slave” surgical instrument has a distal articulation of orientation and grip which is robotically controlled by the manipulator by a mechanical connection interface.

In order to control the robotic manipulator and thus the slave surgical instrument, the surgeon acts on one or more master controller devices, according to a master-slave teleoperation architecture.

In known master devices, buttons are typically included to transmit control signals to the slave surgical instrument.

Other known master devices include articulated structures to support the manipulated part, which are adapted to record or detect movements in space (translations) of such a manipulated part in space. In some prior art examples, the right and left master devices are each provided to be mounted to an attachment of the operative console and supported by a gimbal system adapted to detect the orientation inputs.

Otherwise, master devices of the type mechanically/kinematically not constrained to the operative console (“groundless” or “unconstrained” according to the jargon adopted in this field) are known, i.e., of the “flying master”, which is manipulated by the surgeon in a predeterminable three-dimensional tracking volume. Such unconstrained “flying master” master devices can be suitable for monolateral teleoperation without force feedback.

Where the surgical instrument is provided with an open/close degree of freedom, i.e., gripping/cutting, the master device usually comprises an interface to allow the surgeon to operate such an open/close/gripping/cutting degree of freedom only with his/her fingers.

As shown for example in the prior art document WO-2019-220407 to the same Applicant, an elastic element can be included, specifically a pre-loaded trigger in a cantilevered position between two rigid rods, which is capable of simulating a sort of feedback to the surgeon, stiffening the resistance to closing of the rods, when the opening/closing angle between the rods of the master device is less than a certain predeterminable threshold. A similar solution, showing a master device with differentiated stiffness during closure, is disclosed by U.S. Pat. No. 6,594,552.

Unconstrained master devices are usually equipped with sensors, such as inertial platforms and/or position and/or orientation sensors, such as magnetometers and/or optical markers, to determine the command to be transmitted to the slave surgical instrument.

In some known examples, a magnetic field emitter is provided, which generates a tracking volume in which the position and orientation of two magnetometer-type sensors with six degrees of freedom provided on the body of the master device are tracked, so as to provide closing command signals to the slave device when the detected distance between the sensors is less than a certain threshold.

For example, document WO-2022-175800 to the same Applicant shows an unconstrained master device solution in which the system control unit verifies the existence of predefined geometric relationships between two sensors integrated in the body of the master device itself.

For example, WO-2022-175792 to the same Applicant shows a solution in which the slave surgical instrument is uniquely identified by a virtual control point which is halfway between the two tips (or jaws) of the surgical instrument, which in the master workspace can coincide with the midpoint between the sensors mounted on the rods movable in opening/closing of the body of the master device.

For example, WO-2022-175802 to the same Applicant shows a safety system solution for an unconstrained master device, designed to disable teleoperation in the event of excessive accelerations/speeds detected by the integrated sensors.

The known solutions of a master device not constrained to the operative console mentioned above, although partially advantageous in some respects, are not at all without drawbacks.

In fact, the unconstrained master devices could slip, slide, bump into objects or even fall out of the hand during manipulation, transferring and causing unexpected and unwanted movements of the end-effector of the teleoperated system.

Once grasped, the unconstrained masters could be uncomfortable in manipulation or result in a limited precision mobility of the degrees of freedom thereof during the manipulation thereof.

WO-2019-099584 discloses some further examples of master devices of the unconstrained type, and in particular a solution which includes a sort of handle or grip wearable by the surgeon, equipped with special rings to receive the surgeon's fingers, which can be connected to the opening/closing command operating portion by a spherical joint.

Although partially satisfactory from some points of view, the known examples of master controller devices have a high number of mechanical and electronic components, a high construction complexity, the difficulty of a form which can be sterilized or draped for sterile handling, as well as kinematics which are too complex to limit the simple and free manipulation of the natural degrees of freedom of the hand, compromising control, precision, accuracy and range of motion.

It is thus strongly felt the need to suggest a master device solution with improved ergonomics and safety as compared to known solutions, without resulting in a worsened master-slave teleoperation accuracy or limiting the relative range-of-motion or sterility.

At the same time, the need is felt to provide an intuitive and ergonomic master device to be used which is capable of guaranteeing a satisfactory reliability in the control of a plurality of degrees of freedom of the slave device, which include the open/close degree of freedom i.e., gripping and/or cutting of the surgical instrument of the slave device.

### SUMMARY OF THE INVENTION

It is an object of the present invention to obviate the drawbacks complained of with reference to the prior art.

According to an aspect of the invention, a master controller device comprises a control gripper to control at least the open/close degree of freedom of a slave surgical instrument, a handle, which forms the grip of the master controller device, and a joint between said handle and said control gripper.

The control gripper comprises two interface portions for the surgeon's fingers.

The joint can allow the rotation of the control gripper with respect to the handle about a roll rotation axis which can be generally aligned with the direction of longitudinal development of the control gripper.

In accordance with an embodiment, each interface portion comprises a rigid flap which protrudes cantilevered from the control gripper forming, with a concave surface thereof, a cavity, in which the concave surfaces have concavities facing opposite with respect to the roll rotation axis.

In accordance with an embodiment not necessarily combinable with all the embodiments described above, each interface portion comprises a concave surface delimiting a cavity thereof, in which each concave surface is a conically-developing surface.

In accordance with an embodiment not necessarily combinable with all the embodiments described above, the handle comprises a body forming at least one seat with a surface thereof to rest on the back of the user's hand. Preferably, the surface to rest on the back of the hand is formed by a horn or protrusion of the body of the handle which extends generally oppositely with respect to the control gripper.

By virtue of the suggested solutions, it is possible to respond to the need to improve aspects of instability of the known master controller devices, reducing the number of mechanical and electronic components, as well as the construction complexity, the difficulty of a sterilizable form for sterile handling, and a kinematics which is even too complex to limit the simple and free manipulation of the natural degrees of freedom of the hand.

By virtue of the suggested solutions, the control, precision and accuracy of the master controller device can be improved, simultaneously improving the ergonomics of the handle and of maneuvering.

By virtue of the suggested solutions, a master device is provided, consisting of a limited number of mechanical, electronic and sensor components, as well as simple but effective ergonomics, as well as a solid anti-fall grip which, however, does not compromise the relative freedom of movement and the range-of-motion and/or which has an easily sterilizable or protectable design with sterile cloth, and/or facilitates a controlled, precise and accurate movement even, and in particular, on the yaw and roll degrees of freedom.

### BRIEF DESCRIPTION OF THE DRAWINGS

Further features and advantages of the invention will become apparent from the following description of preferred embodiments, given by way of non-limiting indication, with reference to the accompanying drawings which are briefly described below. Note that references to “an” embodiment in this disclosure do not necessarily refer to the same embodiment, and are to be understood as at least one. Moreover, for reasons of conciseness and reduction of the total number of figures, a certain figure can be used to illustrate the features of more than one embodiment, and not all the elements of the figure can be necessary for a certain embodiment.

FIG. 1 shows an axonometric view of a robotic system for medical or surgical teleoperation, according to an embodiment.

FIG. 2 shows an axonometric view of a slave surgical instrument, according to an embodiment.

FIGS. 3 and 4 show a pictorial perspective view of an unconstrained master controller device, according to an embodiment, held in hand by a surgeon.

FIG. 5 is an axonometric view of a master controller device, according to an embodiment.

FIG. 6 shows a detail of FIG. 5 showing a conically-developing surface of the master controller device, in which a cone is traced in overprint, for clarity.

FIGS. 7A and 7B are raised vertical views of a master controller device, according to an embodiment, in which a spherical joint between the handle and the control gripper is shown.

FIG. 8 is a front view of a master controller device, according to an embodiment.

FIG. 9 is a sectional view of a master controller device made according to the cutting plane indicated by arrows IX-IX in FIG. 8.

FIG. 10 shows a pictorial perspective view of an unconstrained master controller device, according to an embodiment, held in hand by a surgeon.

FIG. 11 is a raised vertical view of the master controller device in FIG. 10.

FIG. 12A is a raised vertical view of a control gripper of a master controller device, according to an embodiment.

FIG. 12B is a sectional view made according to the cutting plane indicated with arrows B-B in FIG. 12A.

FIGS. 13A and 13B show an orientable interface portion and a repositionable interface portion, respectively, according to some embodiments.

FIG. 14 shows a master controller device according to an embodiment in separate parts.

FIG. 15 shows a master controller device according to an embodiment in separate parts.

FIG. 16 shows a pictorial perspective view of a master controller device, according to an embodiment, gripped by a user.

FIG. 17 shows a vertical elevation of a master controller device, according to an embodiment.

FIG. 18 shows a vertical elevation of a master controller device, according to an embodiment.

FIGS. 19 and 20 show a vertical elevation with partially transparent parts, for clarity, of a master controller device according to some embodiments.

FIG. 21 shows a vertical elevation of a master controller device, according to an embodiment.

FIG. 22 shows a master controller device according to an embodiment in separate parts.

### DETAILED DESCRIPTION

Reference throughout this description to “an embodiment” means that a particular feature, structure or function described in relation to the embodiment is included in at least one embodiment of the present invention. Therefore, the formulation “in an embodiment” in various parts of this description do not necessarily all refer to the same embodiment. Moreover, particular features, structures or functions such as those shown in different drawings can be combined in any suitable manner in one or more embodiments.

In accordance with a general embodiment, a master controller device 110 is provided, comprising a control gripper 10 to control at least the open/close degree of freedom GS of a slave surgical instrument 170.

The master device 110 is particularly suitable for a robotic system 100 for surgical teleoperation.

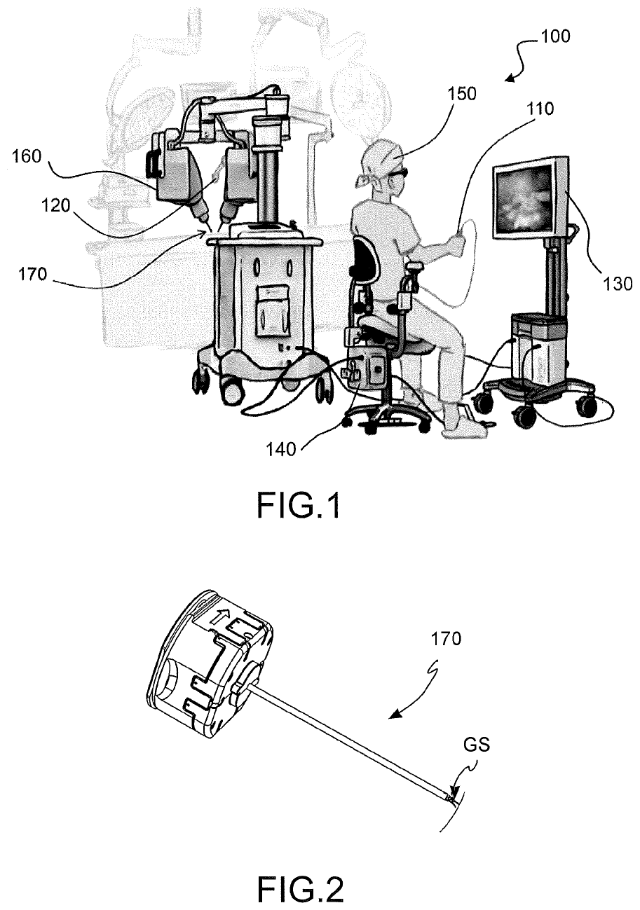

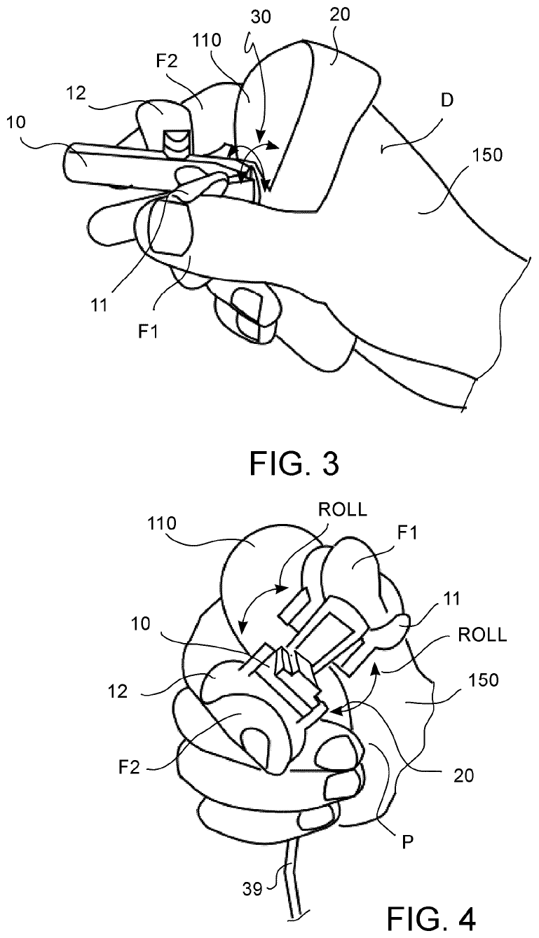

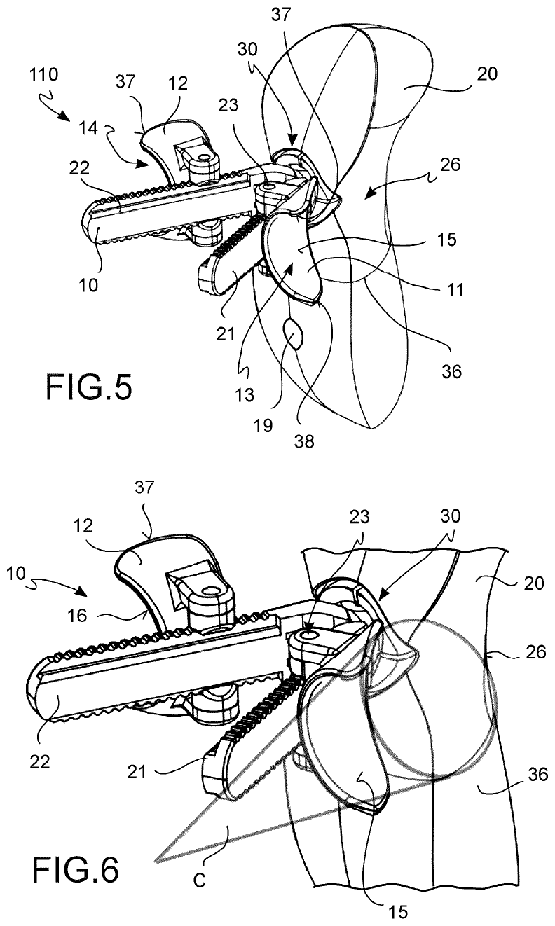

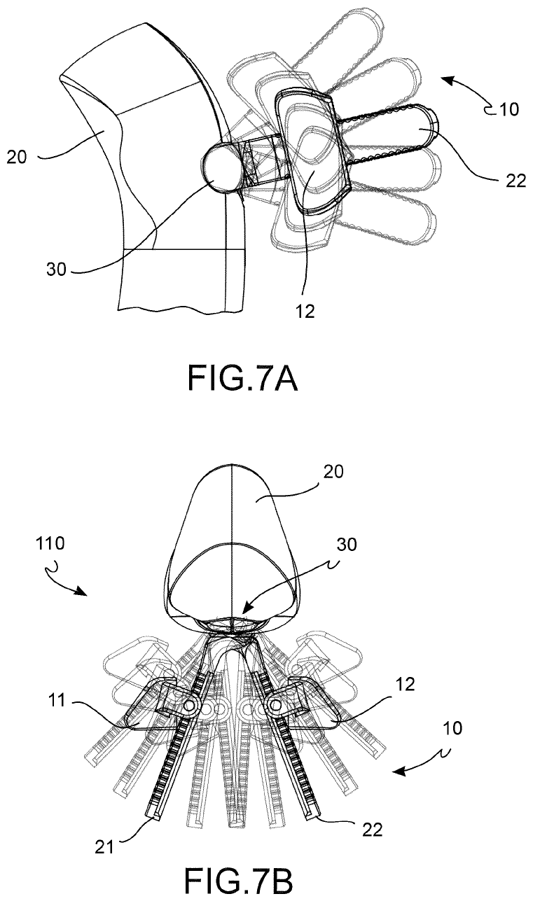

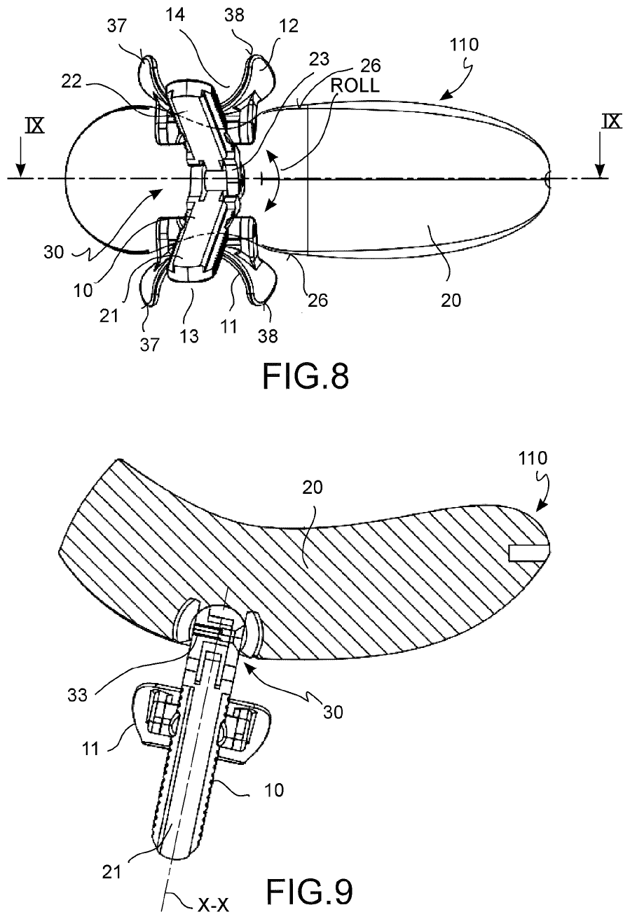

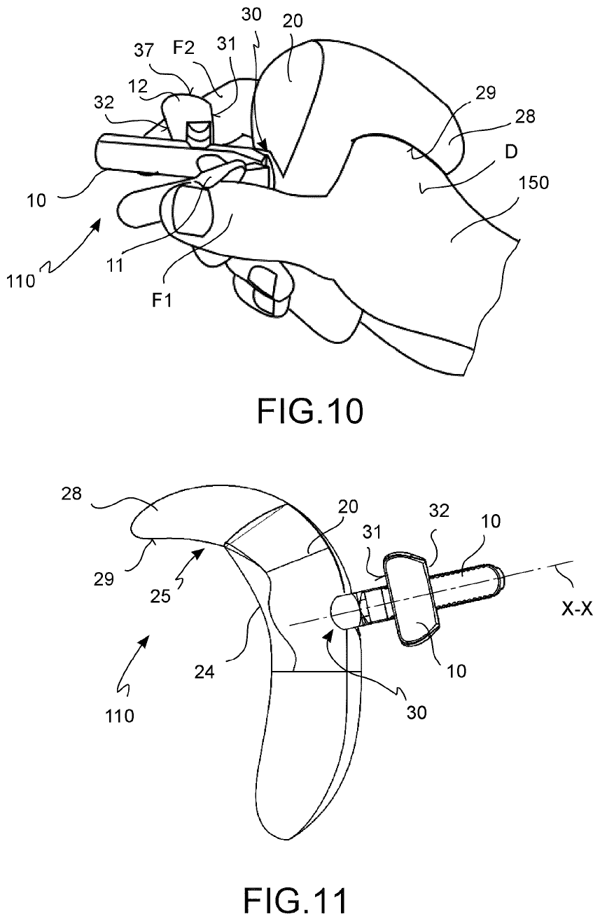

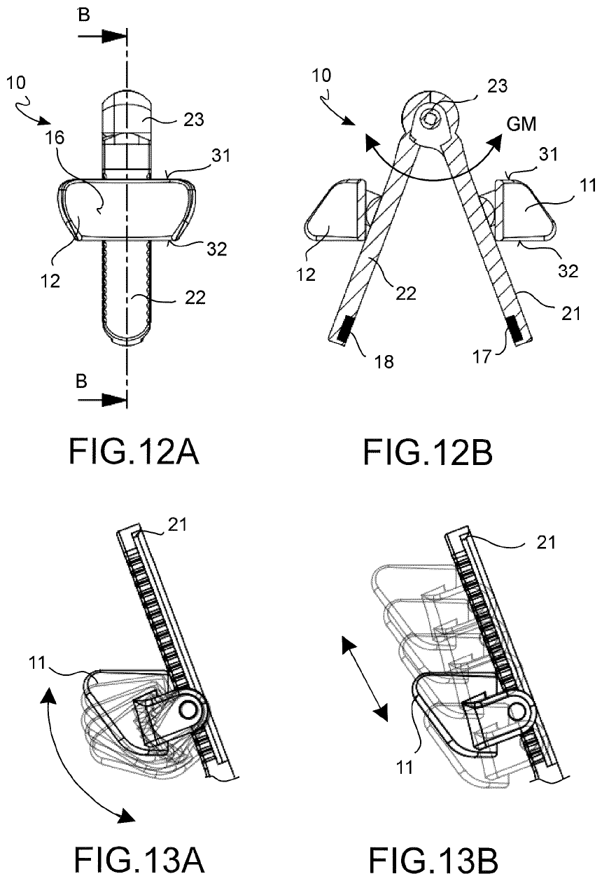

As shown for example in FIG. 1, the robotic system 100 for surgical teleoperation can comprise at least one master controller device 110 and at least one slave device 170 operable under the control of the at least one master controller device 110. A master controller console can be provided, comprising a tracking magnetic field emitter 140 to track the at least one master controller device 110. The master controller console can comprise a screen 130 or display 130 for displaying the surgical site. The slave device 170 comprises at least one slave surgical instrument 170 operable under the control of the at least one master device 110.

Preferably two master devices 110 and two slave surgical instruments 170 are provided, where each master device individually controls a respective slave surgical instrument thereof at least in the respective open/close degree of freedom (e.g., grip, grasping, cutting). The control of the open/close degree of freedom GS of the at least one slave surgical instrument 170 is performed by the control gripper 10 of the at least one master controller device 110.

The at least one slave surgical instrument 170 is preferably connected to a respective at least one robotic manipulator 160 comprising a plurality of motors and actuators capable of actuating the degrees of freedom of the slave surgical instrument 170, for example through a sterile barrier.

The control gripper 10 of the master controller device 110 comprises a first interface portion 11 and a second interface portion 12 for the fingers F1, F2 of a user 150.

The first interface portion 11 and the second interface portion 12 are movable with respect to each other with relative approaching and/or away movement GM, to control the open/close degree of freedom GS of a slave surgical instrument 170. The interface portions 11, 12 can form gripping interface portions for manipulating the open/close degree of freedom GM (“grip”) of the control gripper 10 of the master device 110, for example by imposing, by the fingers F1, F2 of the surgeon 150, a relative approaching force of two rigid parts 21, 22 of the control gripper 10.

Preferably, when under operating conditions, a first finger F1, for example the index finger F1, of a surgeon 150 manipulates said first interface portion 11 and a second finger F2, for example the thumb F2 of the same hand of the surgeon 150, manipulates said second interface portion 12.

Preferably, the first interface portion 11 is provided, on the body of the control gripper 10, opposite to the second interface portion 12, so that the action of the surgeon's fingers F1, F2 is aimed at moving the two opposite interface portions 11, 12 with a relative approaching movement.

In accordance with a preferred embodiment, the control gripper 10 comprises a first rigid part 21 mounting the first interface portion 11 and a second rigid part 22 mounting the second interface portion 12, and a rotational joint 23 between the first rigid part 21 and the second rigid part 22. Preferably, the rotational joint 23 is elastically preloaded in opening so as to counteract the closing force imposed by the user (surgeon) acting on the interface portions 11, 12.

Advantageously, the master controller device 110 further comprises a handle 20, which forms the grip of the master controller device 110, and a joint 30 between said handle 20 and said control gripper 10.

The joint 30 is preferably a rotational joint.

The inclusion of the joint 30 allows a roll rotational degree of freedom ROLL of the control gripper 10 with respect to the handle 20 about a roll rotation axis X-X.

The joint 30 can allow other relative degrees of freedom between the handle and the control gripper.

Preferably, the control gripper 10 extends along a generally longitudinal direction, for example the centerline between the rigid parts 21, 22 of the control gripper 10, in which the roll rotation axis X-X is coincident with the longitudinal direction of the control gripper 10 of the master device 110.

In accordance with an embodiment, the control gripper 10 comprises two rigid parts 21, 22 defining an angle therebetween and the roll axis X-X is substantially coincident with the middle axis between the two rigid parts 21, 22, e.g., elongated rigid rods, although no rigid part 21, 22 can be provided along the extension of said roll axis X-X.

In accordance with a preferred embodiment, said joint 30 between handle 20 and gripper 10 is a rotational joint adapted to allow at least the roll rotation of the control gripper with respect to the handle.

In accordance with a preferred embodiment, said joint 30 between handle 20 and gripper 10 is a spherical joint.

The two interface portions 11, 12 of the control gripper 10 face oppositely with respect to the roll rotation axis X-X.

The user 150, i.e., the surgeon 150, grasps the control gripper from the outside to approach together the parts responsible for controlling the open/close degree of freedom.

In accordance with a preferred embodiment, each interface portion 11, 12 comprises a rigid flap which protrudes cantilevered from the control gripper 10. Said rigid flap extending cantilevered thus forms, with a concave surface 15, 16 thereof, a cavity 13, 14 to receive the fingers F1, F2 of the surgeon 150 in order to facilitate the rolling movement of the control gripper with respect to the handle.

In accordance with an embodiment, each rigid part 21, 22 of the control gripper 10 comprises a flap thereof extending cantilevered away from the roll rotation axis X-X forming said cavity with a concave surface 15, 16 thereof.

The concave surfaces 15, 16 have concavities facing oppositely with respect to the roll rotation axis X-X, i.e., they both face externally with respect to the body of the control gripper 10.

Each rigid flap of the respective interface portion 11, 12 thereof extends cantilevered, forming an attachment portion to the control gripper and at least one cantilevered free end portion.

In accordance with a preferred embodiment, the rigid flap is made in the form of an open concave band defining two free end portions arranged at the edges of the concave surface 15, 16 thereof in a circumferential roll direction (circumferential around the roll axis X-X). Therefore, the cavity 13, 14 is delimited by said two cantilevered free end portions.

In accordance with a preferred embodiment, the cavity 13, 14 of a respective interface portion 11, 12 is a pass-through cavity in the longitudinal direction. When in operating conditions, the surgeon's fingers are thus inserted longitudinally into the respective cavities. Preferably, the term “longitudinally” is intended to indicate the direction of longitudinal development X-X of the control gripper 10, i.e., of the rigid parts 21, 22 thereof, for example the middle axis between the rigid parts 21, 22 of the control gripper.

In accordance with a preferred embodiment, each rigid flap cavity is formed by a respective conically-developing concave surface. In other words, in accordance with this embodiment, the concave surface 15, 16 is a conically-developing surface. When in operating conditions, each finger of the surgeon is received in a respective cavity of the control gripper which is formed by a conically-developing surface. Preferably, only a portion of the surgeon's finger manipulates the conically-developing surface of the cavity, for example only a distal portion of the surgeon's finger.

The inclusion, on each opposite side of the control gripper, of a cavity having a conically-developing handling surface allows the control gripper to be firmly handled because the conically-developing surface of the cavity is tapered, that is, it has a section which narrows due to the conical development thereof, and therefore allows a force coupling with the surgeon's fingertip.

In accordance with a preferred embodiment, the conically-developing surface is formed by an open concave frusto-conical band. Preferably, the frusto-conical band is open in a transverse direction with respect to the longitudinal direction, i.e., the frusto-conical cavities mutually face in transversely opposite directions. Each cavity 13, 14 of the respective interface portion 11, 12 is thus made from an open frusto-conical band, which faces a conically-developing surface 15, 16 thereof at the respective finger F1, F2 of the surgeon. The inclusion of a frusto-conical band which is open allows an easy transverse insertion of the respective finger of the surgeon, without requiring a longitudinal insertion of the finger.

The open frusto-conical band can form a first proximal edge 31 and a second distal edge 32 which longitudinally delimit the extension of the conically-developing band.

The open frusto-conical band can be cut, forming inclined edges. The edges or vertices can be rounded.

The conically-developing surface 15 or 16 can be made with a molded plastic body (e.g., polyethylene, polyester, polyurethane).

Preferably, the cavity 13 formed by the conically-developing surface 15 of the first interface portion 11 is tapered, i.e., narrows, along the same direction as the cavity 14 formed by the conically-developing surface 16 of the second interface portion 12. For example, both narrow distally.

In accordance with an embodiment, the first interface portion 11 and/or the second interface portion 12 are repositionable in the longitudinal direction along the body of the control gripper 10. To this end, a prismatic joint can be provided between an interface portion 11 or 12 and the body of the control gripper 10, for example a respective rigid part 21 or 22. For example, a rigid part 21 or 22 of the body of the control gripper 10 acts as a guide or track and the respective interface portion 11 or 12 comprises a slider slidably mounted on the rigid part.

Alternatively or additionally, the first interface portion 11 and/or the second interface portion 12 is reorientable to accommodate the fingers F1, F2 of the user 150 in a variety of configurations. In order to adjust the orientation, a rotational joint 33 can be provided between an interface portion 11 or 12 and the body of the control gripper 10, for example a respective rigid part 21 or 22. Adjusting the orientation and/or position preferably does not change the opposition geometry relationship between the two handling cavities 13, 14 for receiving the fingers.

Preferably, the master controller device 110 further comprises a handle 20, which forms the grip of the master controller device 110. The handle 20 is mounted on the control gripper 10. In accordance with one embodiment, a spherical joint is provided between the control gripper 10 and the handle 20.

The handle 20 can have a body made of rubber, gel or other plastic material to allow a comfortable grip for the surgeon and can be made by molding. The handle body can be internally hollow to house electronic chips and/or wirings therein for controlling the master device.

In accordance with a preferred embodiment, the handle 20 comprises a body forming a seat 25 with a surface 29 thereof to rest on the back D of the user's hand 150. Preferably, the surface 29 to rest on the back of the hand is formed by a horn 28 or protrusion 28 of the body of the handle 20 which extends oppositely with respect to the control gripper 10. When in operating conditions, the surgeon thus grasps the handle 20 and with his/her fingers F1, F2 maneuvers the control gripper 10, which can extend frontally, while the horn 28 extends rearwards to rest on the back D of the surgeon's hand, for example in the area between the thumb F2 and the index finger F1.

As shown for example in FIG. 17, the handle 20 can comprise two horns 28 or protrusions 28 with a respective surface 29 to rest on the back D of the surgeon's hand, in which the two horns 28 or protrusions 28 are arranged on opposite sides of the grip formed by the handle 20. In other words, the handle 20 can comprise a grip portion or segment 36 delimited by two horns or protrusions 28. The grip portion or segment 36 can be shaped to improve the gripping/grasping ergonomics of the surgeon. For example, the grip portion or segment can have a surface processing aimed at increasing grip or adhesion in the hand, such as knurling. For example, the grip portion 36 can be narrower, that is, it can have a smaller cross-sectional area than other portions of the same handle 20.

Preferably, the horn 28 or protrusion 28 and in particular the surface 29 is located at a higher level than the joint 30 for the connection with the control gripper 10. The support with the back of the hand is thus higher than the position determined by the articulated joint or by the connection of said rigid parts of the control gripper with the handle. In other words, the handle has a main extension along a direction of development thereof, where the surface 29 is at a certain height or level along said direction of development of the handle which is more extreme than the height or level along said direction of development at which the joint 30 for the connection with the control gripper is provided. Preferably, when in operating conditions, the surface 29 of said at least one horn 28 or protrusion is at a higher level than the joint 30 for the connection with the control gripper 10.

Transversely, the body of the handle 20 can comprise two opposite flanks 26 which narrow forming a throat. The grip of the handle 20 by the surgeon is thus facilitated. In accordance with an embodiment, the transverse throat is arranged close to said interface portions 11, 12 so that the fingers F1, F2 of the surgeon manipulating the control gripper 10 with their distal portions have a respective proximal segment thereof resting on the surface of the throat of the opposite transverse flanks 26 of the handle body.

The handle 20 can have a main extension in a direction of development thereof which is preferably not aligned with the longitudinal direction of the control gripper, forming an angle therewith. Where, for example, a spherical joint 30 is provided between the handle and the control gripper, a solid angle is formed.

This allows further improving the ergonomics of gripping and maneuvering the master device 110.

In accordance with an embodiment, the electronics necessary for tracking the master device 110 are entirely contained in the control gripper 10. It is thus possible to concentrate the active parts in the control gripper, leaving the ergonomic function to the handle 20, allowing the electrically passive and thus not powered handle to be made, resulting in a reduction in the manufacturing time and costs of the handle and a rationalization of the production process of the master device 110 as a whole (control gripper).

In accordance with an embodiment, the control gripper 10 comprises a first tracking sensor 17 associated with the first interface portion 11 and a second tracking sensor 18 associated with the second interface portion 12, for detecting information aimed at controlling at least the open/close degree of freedom of the slave surgical instrument. Preferably, each tracking sensor 17, 18 is a position and orientation sensor with six degrees of freedom. The handle 20 can comprise one or more control buttons 19 to control an additional function of the slave device 170 (with respect to controlling the opening/closing of the slave device 170), i.e., to transmit command signals to the slave device 170.

Where the control gripper comprises a first rigid part 21 mounting the first interface portion 11 and a second rigid part 22 mounting the second interface portion 12, and a rotational joint 23 between the first rigid part and the second rigid part, the rotational joint 23 can be arranged between the interface portions 11, 12 and the spherical joint 30 for the connection with the handle 20.

The inclusion of such a master controller device favors the roll movement about the longitudinal axis of the control gripper 10 to roll the control gripper with respect to the handle.

Each cavity 13, 14 can have at least one degree of freedom with respect to the rigid part to which it is mounted. The at least one degree of freedom allows the movement of the fingers to be followed during handling, improving the surgeon's sense of control.

In order to accommodate different sized hands and fingers, each interface portion 11, 12 can be made in a separate piece with respect to the respective rigid part of the control gripper which can be released, translationally moved and reattached in a variety of configurations.

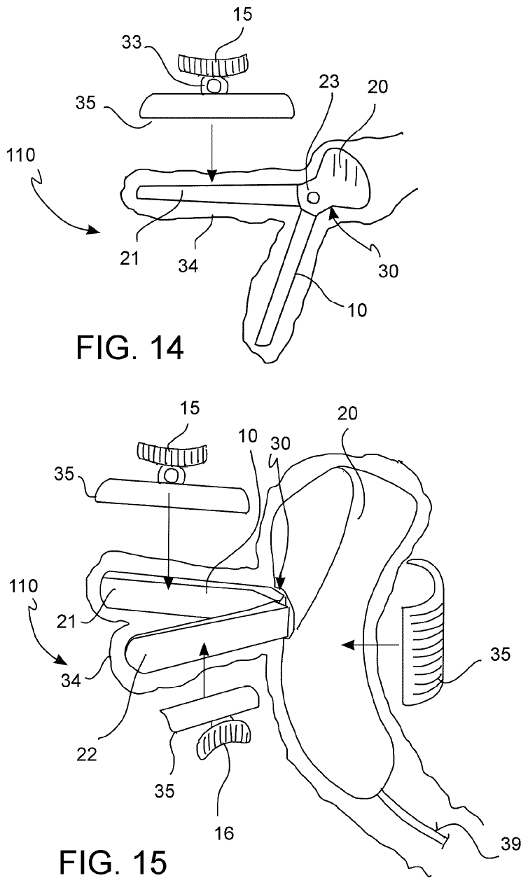

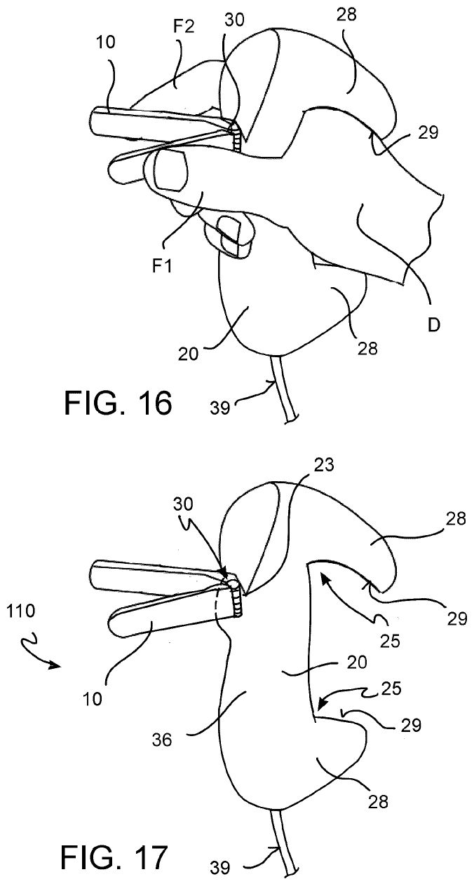

As shown for example in FIG. 14, each interface portion 11, 12 can be mounted on a sterile clip 35 which in turn can be coupled integrally with each of said rigid parts 21, 22 of the control gripper 10, through a sterile drape 34 to cover the control gripper 10 and if necessary, also the handle 20. The sterile clip 35 is preferably capable of snap-fitting with the body of the rigid part 21, 22 of the control gripper 10 through the sterile drape 34. As shown for example in FIG. 15, a sterile clip 35 can also be mounted on the handle 20, for example snap-fit. The conically-developing surfaces 15, 16, where the fingertips rest, can be part of a sterile clip 35 which snap-fits to the rigid part of the master device.

The tracking sensors 17, 18 can be integrally mounted to the first and second interface portions 11, 12, respectively, and can be configured to detect both the open/close degree of freedom GM of the control gripper 10 and the orientation degree of freedom of the control gripper 10, which for example translates into activation of the degree of freedom of yaw of the slave surgical instrument 170, which can comprise an articulated cuff.

Preferably, the tracking sensors 17, 18 are mounted integrally to the rigid parts 21, 22 of the control gripper 10 and each interface portion 11, 12, with the cantilevered flap thereof, is movable with respect to the sensors 17, 18 because it is movable with respect to the respective rigid part 21, 22 thereof. The workspace is thus extended in the roll direction because the fingers can perform large movements which are not necessarily entirely tracked and transmitted to the slave device 170.

One or more control buttons 19 can be provided on the handle to control, for example, freezing some degrees of freedom (“HOLD MODE”), such as enabling/disabling the transmission of pure translation commands to the slave device 170 and/or controlling and modifying the scaling factor of master-slave teleoperation. At least one control button 19 can be located onto said at least one horn 28 or protrusion 28. The arrangement of the buttons 19 can be chosen so that they are all accessible by the hand of the surgeon gripping the master controller device or they can be appropriately located in portions of the handle 20 which are not accessible to impose caution in the activation thereof.

The control gripper 10 can be made detachable, i.e., removable from the handle 20, to allow for example the replacement of the handle 20. For example, the control gripper 10 can comprise a male connection element and the handle can delimit a female connection element, said male and female connection elements forming said rotational joint 30, preferably a spherical joint 30 between the handle 20 and the control gripper 10.

The joint 30 can be a rotational joint, a joint, a spherical joint, or the like.

The master controller device 110 is preferably a master controller device of the type not constrained to the operative console, i.e., of the “flying master” freely movable in the tracking field thereof.

The handle 20 of the master controller device can be protected by a sterile cloth or drape, and then assembled to the control gripper 10.

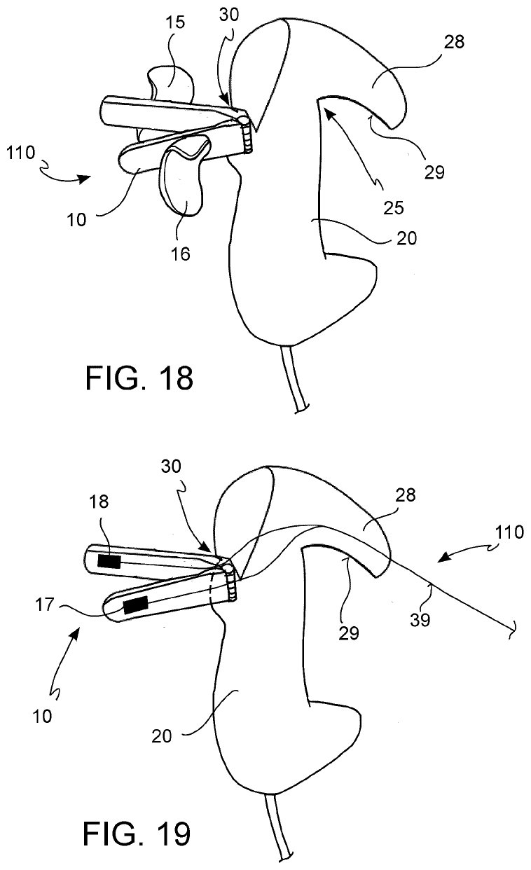

The handle 20 of the master controller device can be made as a sterilizable component which is assembled to the control gripper 10, as shown for example in FIG. 18. For example, the handle 20 is made of silicone material and lacks active parts.

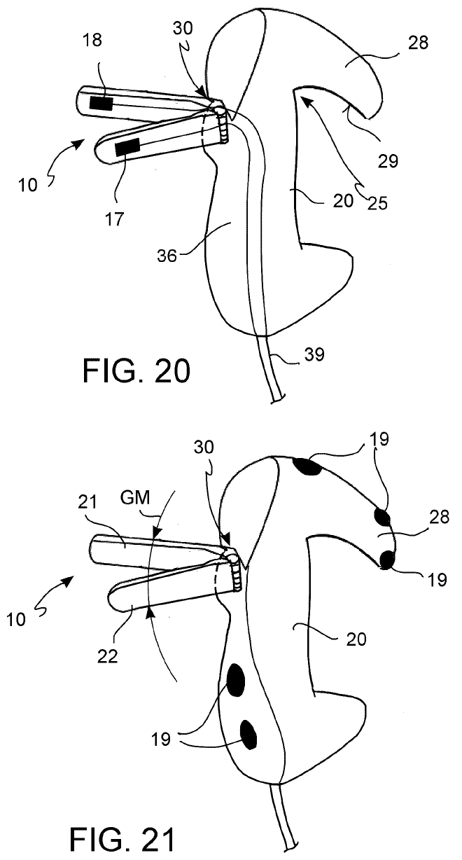

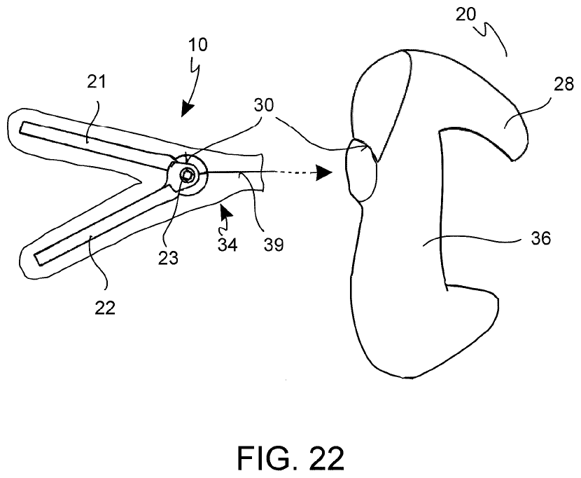

The wirings 39 of the sensors 17, 18 can pass through a passage or channel provided inside the handle body, as shown for example in FIG. 19 or FIG. 20.

The tracking sensors 17, 18 can be optical markers which cooperate with an image acquisition system 120 (e.g., acquisition chips).

By virtue of the embodiments described above, provided jointly or separately in particular embodiments, it is possible to meet the aforementioned needs, obtaining the aforementioned advantages, and in particular:

the concave flaps of the control gripper provided in combination with the handle and with the roll joint between the control gripper and the handle allow the surgeon to produce a roll rotation force of the gripper with respect to such a handle with is more controlled, which therefore translates into a more precise activation of the slave device;

the inclusion of said cavities defined by the interface portions favors the roll manipulation of the control gripper and, preferably, each cavity is designed to receive a finger of the surgeon;

an improved ergonomics of the master device intended to be held by the surgeon is allowed, particularly if the master device is a device of the flying master, i.e., not mechanically constrained to the console and movable in three-dimensional space substantially freely when within the tracking field thereof (magnetic and/or optical, for example);

at the same time, a master device solution is provided, which is designed to minimize the risk of the master device inadvertently falling from the surgeon's hand, particularly if the master device is a flying master device;

an extension of the roll workspace is allowed, since some movements of the surgeon's fingers in a circumferential direction about the roll axis cannot be tracked and therefore are not transmitted to the slave device associated with the master device;

in particular, the inclusion of at least one roll rotational degree of freedom between each rigid flap and the respective elongated rigid part thereof, rotation around the longitudinal development axis of the elongated rigid part itself, allows maximizing the aforesaid roll workspace;

a master device solution is provided, which is sterilizable, i.e., it can be protected by a sterile drape without causing the loss of functionality and/or reliability when in operation;

the inclusion of said handle equipped with said at least one horn allows improved ergonomics, as well as an anti-fall function ensuring a contact point with the hand even when the fingers are not fully gripped on the handle at the top or bottom or both;

the inclusion of said handle equipped with flanks close to the control gripper allows implementing the complete closure of the control gripper as well as implementing the roll rotation thereof with respect to said handle without limitations of range-of-motion, ergonomics, as well as without imposing discomfort;

the inclusion of conically-developing concave surfaces allows firmly receiving the surgeon's fingers.

It is well understood that the combinations of features of the appended claims form an integral part of the present description.

Those skilled in the art may make several modifications and adaptations to the above-described embodiments and may replace elements with functionally equivalent elements without, however, departing from the scope of the appended claims.

LIST OF REFERENCE SIGNS

| | |
|---|---|
| 10 | Master device control gripper |
| 11 | First gripper interface portion |
| 12 | Second gripper interface portion |
| 13 | First gripper interface cavity |
| 14 | Second gripper interface cavity |
| 15 | First concave surface of the first interface portion |
| 16 | Second concave surface of the second interface portion |
| 17 | First tracking sensor |
| 18 | Second tracking sensor |
| 19 | Control button |
| 20 | Master device handle |
| 21 | First rigid part of the gripper |
| 22 | Second rigid part of the gripper |
| 23 | Rotational joint between the rigid gripper parts |
| 24 | Handle surface for the hand palm |
| 25 | Handle throat |
| 26 | Handle flank |
| 28 | Handle horn or protrusion |
| 29 | Horn surface to rest on the back of the hand |
| 30 | Master device joint |
| 31 | First proximal edge of the concave surface |
| 32 | Second distal edge of the concave surface |
| 33 | Rotational joint of the interface portion |
| 34 | Sterile drape or sterile cloth |
| 35 | Sterile clip |
| 36 | Handle grip portion or segment |
| 37 | First free end portion of the flap |
| 38 | Second free end portion of the flap |
| 39 | Wiring |
| 100 | Robotic system for medical or surgical teleoperation |
| 110 | Master controller device |
| 120 | Image acquisition system |
| 130 | Display |
| 140 | Tracking device, or tracking field emitter |
| 150 | Operator, or user, or surgeon |
| 160 | Robotic manipulator |
| 170 | Slave device, or slave surgical instrument |
| F1, F2 | Fingers of the hand, e.g., index finger and thumb |
| C | Conical development |
| P | Palm of the hand |
| D | Back of the hand |
| GM | Opening/closing of the master device gripper |
| GS | Opening/closing or gripping or cutting of the slave device |
| X-X | Roll rotation axis of the master device |

1. A master controller comprising:
a control gripper to control at least an open/close degree of freedom of a slave surgical instrument;
a handle;
a joint between said handle and said control gripper, said joint allowing a roll rotational degree of freedom of the control gripper with respect to the handle about a roll rotation axis;
wherein:
the control gripper comprises two interface portions facing opposite relative to the roll rotation axis;
each interface portion comprises a rigid flap that protrudes cantilevered from the control gripper, forming a concave surface comprising a cavity;
the concave surfaces have respective concavity facing opposite relative to the roll rotation axis.

2. The master controller of claim 1, wherein each concave surface is a conically developing surface.

3. The master controller of claim 2, wherein the cavity formed by the conically developing surface of the first interface portion is tapered along a same direction as the cavity formed by the conically developing surface of the second interface portion.

4. The master controller of claim 1, wherein each concave surface comprises an open, frustoconical band.

5. The master controller of claim 1, wherein the cavity of each interface portion is a pass-through cavity in a longitudinal direction.

6. The master controller of claim 1, wherein the first interface portion and/or the second interface portion is repositionable in a longitudinal direction along the body of the control gripper.

7. The master controller of claim 1, wherein each interface portion is reorientable with respect to the control gripper.

8. The master controller of claim 1, wherein the control gripper comprises two rigid parts that are movable with respect to one another to control said open/close degree of freedom, and the interface portions are mounted onto said two rigid parts, respectively.

9. The master controller of claim 8, wherein a rotational joint is between the two rigid parts of the control gripper, wherein, said rotational joint is located between the joint to connect to the handle and the two interface portions of the control gripper.

10. The master controller of claim 8, comprising tracking sensors integrally mounted to said rigid parts of the control gripper, wherein each interface portion is movable with respect to said tracking sensors.

11. The master controller of claim 1, wherein said joint between the control gripper and the handle that makes said roll rotational degree of freedom is a spherical joint.

12. The master controller of claim 1, wherein the handle comprises a body forming at least one seat with a surface to rest on a back of a hand of a user.

13. The master controller of claim 12, wherein the surface to rest on the back of the hand is made by a horn or protrusion of the body of the handle that extends opposite with respect to the control gripper.

14. The master controller of claim 1, wherein the handle has a body comprising two opposite flanks that narrows forming a throat to receive a user's fingers, said throat being arranged proximate said interface portions and/or said joint.

15. The master controller of claim 1, wherein the handle comprises a control button to control an additional function in addition to control of the open/close degree of freedom; wherein said at least one control button is located on said horn.

16. The master controller of claim 1, comprising a sterile drape to cover the control gripper, and two sterile clips snap-fit to the control gripper through the sterile drape; and
wherein each interface portion comprises a rigid cantilevered flap being mounted to a respective sterile clip of the two sterile clips.

17. The master controller of claim 1, comprising a sterile drape to cover the handle and a sterile clip that snap-fits to said handle through said sterile drape.

18. The master controller of claim 1, wherein said handle is a sterile element that couples to said control gripper.

19. The master controller of claim 1, wherein the first interface portion and/or the second interface portion is repositionable in the longitudinal direction along the body of the control gripper, and along a respective rigid part thereof.

20. The master controller of claim 1, wherein the handle comprises a control button to control an additional function in addition to the control of the open/close degree of freedom, comprising control of the scaling factor of master-slave teleoperation and/or the freezing of some degrees of freedom of the slave device; wherein said at least one control button is located on said horn.
